(19) United States
(12) Patent Application Publication
(10) Pub. No.: US 2014/0261436 A1
(43) Pub. Date: Sep. 18, 2014

(54) PATIENT INTERFACE THAT ENGAES THE SIDES OF THE HEAD

(71) Applicant: KONINKLIJKE PHILIPS N.V., EINDHOVEN (NL)

(72) Inventors: Jonathan Paul McCaslin, Renfrew, PA (US); Adam Michael Neff, JR., Oakmont, PA (US); Jeffry Huth, Delmont, PA (US); Susan Mals, Pittsburgh, PA (US)

(21) Appl. No.: 14/353,801

(22) PCT Filed: Oct. 18, 2012
(86) PCT No.: PCT/IB2012/055685

§ 371 Date: Apr. 24, 2014

Related U.S. Application Data
(60) Provisional application No. 61/551,952, filed on Oct. 27, 2011.

(51) Int. Cl. A61M 16/06; A61M 16/00

(52) U.S. Cl. CPC A61M 16/0683; A61M 16/0057; A61M 16/0622; A61M 16/0666; A61M 16/0051; A61M 16/0006
USPC 128/205.25

(57) ABSTRACT

An improved patient interface (4) having a cushion (12) and a headgear (16), with the headgear having a pair of engagement elements (24) that each engage the patient with an engagement force, and with the engagement forces being directed generally toward one another. The engagement forces result in frictional forces between the headgear and the patient which resist the cushion from becoming disengaged with the patient. In one embodiment, the engagement elements each include a circumferential brace (44) that engages the head and extends about the ears. In another embodiment, the engagement elements engage the ears of the patient. In a free state, at least a portion of the headgear is of a spiral configuration which makes the patient interface collapsible and relatively easy to pack, such as for traveling.

### CROSS-REFERENCE TO RELATED APPLICATIONS

This patent application claims the priority benefit under 35 U.S.C. §119(e) of U.S. Provisional Application No. 61/551,952 filed on Oct. 27, 2011, the contents of which are herein incorporated by reference.

### BACKGROUND OF THE INVENTION

1. Field of the Invention

The present invention pertains to a patient interface for delivering a flow of breathing gas to a patient and, in particular, to an improved patient interface that engages a head of a patient with a pair of engagement forces that are oriented generally toward the patient and toward one another.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a respiratory cushion device including a mask apparatus that is typically secured on the face of a patient by a headgear assembly. The mask apparatus may be, without limitation, a nasal mask that covers the patient's nose, a nasal cushion having nasal prongs that are received within the patient's nares, a nasal/oral mask that covers the nose and mouth, or full face mask that covers the patient's face. It is known to maintain such devices on the face of a wearer by a headgear having one or more straps adapted to fit over/around the patient's head. Because such respiratory cushion devices are typically worn for an extended period of time, it is important for the headgear to maintain the mask apparatus in a desired position while doing so in a manner that is comfortable to the patient.

While many mask apparatuses have been generally been effective for their intended purposes, they have not been without limitation. For instance, patients often complain that the mask apparatuses are not as comfortable as they would desire. Also, it is understood in the art that the considerations of patient comfort, ease of installation and use, and reliability of providing the flow of breathing gas to the patient are often mutually in competition with one another. Patients have further complained that such mask apparatuses are cumbersome to travel with due to their unusual shape. It thus would be desirable to provide an improved apparatus that provides a flow of breathing of gas to a patient.

### SUMMARY OF THE INVENTION

In certain embodiments, the general nature of the invention can be stated as including a patient interface that is structured to deliver a flow of breathing gas to the airways of a patient. The patient interface can be generally stated as including a cushion and a headgear. The cushion is structured to be in fluid communication with the airways of the patient and to supply the flow of breathing gas to the airways of the patient. The headgear can be generally stated as including a frame and a pair of engagement elements. The pair of engagement elements are disposed on the frame and are structured to be engaged with the patient. The engagement elements when engaged with the patient are each structured to apply an engagement force in a direction generally toward the patient, with the engagement forces being directed generally toward one another. The cushion is disposed on the frame.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a perspective view of an improved patient interface in accordance with a first embodiment of the present invention;

FIG. 2 is a view similar to FIG. 1, except depicting the patient interface donned by a patient;

FIG. 3 is a schematic depiction of an electronic apparatus of the patient interface of FIGS. 1 and 2;

FIGS. 4-6 diagrammatically depict the patient interface of FIGS. 1 and 2 moving from a donned state in a condition situated on the patient to a free state in a condition removed from the patient;

FIG. 7 is a perspective depiction of an improved patient interface in accordance with a second embodiment of the present invention;

FIG. 8 is a sectional view as taken along line 8-8 of FIG. 7 and depicting the sectional portion of the patient interface of FIG. 7 situated on the patient;

FIG. 9 is a depiction of an improved patient interface in accordance with a third embodiment of the present invention; and

FIG. 10 is a view of an improved patient interface in accordance with a fourth embodiment of the present invention.

Similar numerals refer to similar parts throughout the specification.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

An improved patient interface 4 in accordance with a first embodiment of the present invention is indicated generally in FIGS. 1, 2, and 4. In particular, patient interface 4 is depicted in FIGS. 1, 2, and 4 in a donned state, which is in a condition wherein it is donned by patient 8, although it is noted that patient 8 is not depicted in FIG. 1 for purposes of clarity. As will be set forth in greater detail below in conjunction with FIGS. 5 and 6, patient interface 4 is movable between the donned state (FIGS. 1, 2, and 4) and a free state (FIG. 6), which is a condition removed from patient 8.

Patient interface 4 can be said to include a cushion 12 and a headgear 16 that are connected together. More particularly, cushion 12 is situated on headgear 16, and headgear 16 is structured to be situated on patient 8. As will be set forth in greater detail below, headgear 16 is advantageously configured to engage patient 8 and, in so doing, retain cushion 12 in fluid communication with the nose of patient 8 in order to provide a flow of breathing gas to the airways of patient 8. In this regard, it is understood that patient interface 4, during use, will be connected with a source of breathing gas (not expressly depicted herein) that provides a flow of breathing gas to patient interface 4 for reception in the breathing passages of patient 8.

While cushion 12 is depicted herein as being a pillow style nasal cushion having a pair of nozzles that are received in the nares of the nose of patient 8, it is understood that the depicted cushion 12 is merely one example of a cushion from among a wide variety thereof that can be employed in patient interface 4. It is therefore to be understood that any type of cushion such as that depicted herein or an alternative cushion such as a nasal cushion, a nasal/oral cushion, a cradle-style nasal cushion, or any other type of structures that can be situated in fluid communication with the airway of patient 8 can be employed as cushion 12.

Headgear 16 can be said to include a frame 20, a first engagement element 24, and a second engagement element 28. First and second engagement elements 24 and 28 are structured to engage patient 8, as can be understood from FIGS. 2 and 4.

Frame 20 can be said to include a base 32, a first leg 36, and a second leg 40. As can be understood from FIG. 1, first and second legs 36 and 40 can be said to extend in a common direction, i.e., in generally the same direction, away from base 32. Cushion 12 is disposed on base 32. First engagement element 24 is mounted on first leg 36 at an end thereof opposite its connection with base 32, and second engagement element 28 is mounted on second leg 40 at an end thereof opposite its connection with base 32. When patient interface 4 is donned by patient 8, cushion 12 can be said to be situated generally in the nasal region of patient 8, and first and second legs 36 and 40 can each be said to extend generally between the nasal region and an otological region of patient 8.

First engagement element 24 can be said to include a circumferential brace 44 having an opening 48 formed therein. Second engagement element 28 can likewise be said to include a circumferential brace 52 having an opening 56 formed therein. As can be understood from FIG. 2, circumferential braces 44 and 52 are structured to be engaged with the head of patient 8 and can be said to each extend about the base of the ear, i.e., at the region where the ear connects with the head. At least a portion of the ears are received in and through openings 48 and 56. While circumferential braces 44 and 52 are depicted as being generally annular in shape, and while openings 48 and 56 are likewise depicted as being generally circular in shape, it is noted that such shapes are intended solely for purposes of simplicity of disclosure. It is expressly noted that in other embodiments, openings 48 and 56 may be configured to more closely follow the shape of the base of the ears, such as by being generally of a “D” shape or other appropriate shape, with circumferential braces 44 and 52 being correspondingly shaped. Other appropriate shapes will be apparent to one of ordinary skill in the art.

As can be understood from FIGS. 1 and 4, circumferential braces 44 and 52 each engage the head of patient 8 with an engagement force, such as indicated with the numerals 60 and 64, which retains patient interface 4 on patient 8. That is, when first and second engagement elements 24 and 28 are pulled away from one another, such as during installation of patient interface 4 on patient 8, frame 20 is elastically deformed, meaning that frame 20 is deformed within its own elastic limits. When first and second engagement elements 24 and 28 are released and caused to become engaged with the head of patient 8, therefore, the residual elastic forces within frame 20 result in first and second engagement elements 24 and 28 engaging the head of patient 8 with engagement forces 60 and 64, respectively, which are each directed toward patient 8 and which are also directed generally toward one another. Frame 20 can be formed of any of a variety of appropriate materials such as spring steel, appropriate plastics having a sufficient degree of resilience, and other appropriate materials.

Engagement forces 60 and 64 applied to patient 8 result in a number of frictional forces between patient interface 4 and patient 8, with one of the frictional forces being indicated generally at the numeral 66. Frictional force 66 in FIG. 1 occurs between the skin of patient 8 and second engagement element 28 and results from the application of engagement force 64 by second engagement element 28 to the skin as a result of the residual elastic forces within frame 20.

More particularly, and as will be apparent to one of ordinary skill in the art, engagement forces 60 and 64 applied to the skin of patient 8 generate frictional forces that resist movement of first and second engagement elements 24 and 28 with respect to the skin. It is understood that when patient interface 4 is donned by patient 8, cushion 12 is engaged with or is otherwise in fluid communication with the airways of patient 8. The frictional forces that result from engagement forces 60 and 64 advantageously resist movement of patient interface 4 in any direction that would be away from cushion 12 being maintained in fluid communication with the airways of patient 8. As such, while only the individual friction force 66 is depicted herein for purposes of simplicity to depict resistance to one exemplary direction of disengagement of cushion 12 from patient 8, it is to be understood that various frictional forces result from engagement forces 60 and 64 and which resist cushion 12 from becoming disengaged from patient 8.

By way of example, therefore, and as depicted in FIG. 1, friction force 66 represents a frictional force applied to second engagement element 28 by the skin of patient 8 and resists a disengagement movement of cushion 12 in a direction away from the nose of patient 8, such as may be due to gravity. Such a disengagement movement could be characterized as the motion of patient interface 4 in a direction generally counter-clockwise from the perspective of FIGS. 1 and 2 (it being reiterated that FIGS. 1 and 2 are perspective views rather than side elevational views where such a disengagement motion would be more explicitly counter-clockwise). Such generally counter-clockwise movement from the perspective of FIGS. 1 and 2 typically would be movement generally about a center (not expressly identified in FIGS. 1 and 2) of openings 48 and 56. Also, it can be seen from FIG. 1 that friction force 66 is spaced from the center point of opening 56, with the result that first friction force 66 applies a torque to second engagement element 28 and to patient interface 4. Such torque resists, for instance, pivoting movement of cushion 12 out of the nares (by way of example) of the nose of patient 8.

It is reiterated, however, that friction force 66 is only one of a variety of frictional forces that result from engagement forces 60 and 64. That is, the various frictional forces that result from engagement forces 60 and 64 together resist any of a variety of movements in any of a variety of directions that otherwise might permit cushion 12 to become disengaged from patient. The resistance to such disengagement is advantageous and promotes the reliability of patient interface 4 in providing a flow of breathing gas to patient 8.

As can be understood from FIG. 3, headgear 16 further includes an electronic apparatus 67. Electronic apparatus 67 is, in the exemplary embodiment presented herein, disposed on frame 20. Electronic apparatus 67 comprises a processor apparatus 68 and an input/output apparatus 70 connected together. In the exemplary embodiment depicted herein, processor apparatus 68 and input/output apparatus 70 are largely embedded within the interior of frame 20 and thus are not necessarily visible from the perspective of FIGS. 1 and 2. Electronic apparatus 67 enables patient interface 4 to provide additional features and thus comfort to patient 8, as will be set forth in greater detail below.

As can be seen in FIG. 3, processor apparatus 68 can be said to include a processor 72 and a memory 74 operatively connected with one another. As is generally understood, memory 74 has stored therein one or more instructions in the form of routines that are executable on processor 72 to cause processor 72 to control various operations of patient interface 4. Memory 74 can be any of a wide variety of storage devices such as RAM, ROM, EPROM, EEPROM, FLASH, and the like without limitation.

Input/output apparatus 70 can be said to include a pair of audio transducers in the form of a microphone 76 and a speaker 78, both of which are connected with processor apparatus 68. Input/output apparatus 70 further includes a wireless transceiver 80 that can communicate wirelessly with a remote wireless device using any of a wide variety of well known wireless protocols such as IEEE 802.11 and the like without limitation. Speaker 78 can be employed to generate an audible output responsive to a signal generated by processor apparatus 68. Such a signal can result from music, by way of example, stored in memory 74 or streamed via wireless transceiver 80 to processor apparatus 68. In one embodiment, one of the routines stored in memory 74 is a noise cancellation routine wherein microphone 76 detects sounds in the vicinity of first and second engagement elements 24 and 28 and generates a resultant signal which is communicated to processor apparatus 68 and is subjected to processing by the noise cancellation routine. The noise cancellation routine then causes processor apparatus 68 to generate a noise cancellation signal which is communicated to speaker 78 to cause speaker 78 to provide as an audible output a noise cancellation output that is responsive to the signal generated by microphone 76. Other uses of input/output apparatus 70 will be apparent to one of ordinary skill in the art, such as the generation of white noise, other audible signals, etc., any of which can provide enhanced comfort to patient 8 and assist patient 8 in sleeping.

As can be understood from the exemplary embodiment of FIGS. 4-6, patient interface 4 is movable between a donned state, such as is depicted generally in FIGS. 1, 2, and 4, and a free state such as is depicted generally in FIG. 6. The donned state is a condition in which patient interface 4 is donned by patient 8 and wherein first and second engagement elements 24 and 28 are engaged with patient 8. The free state is a condition in which patient interface 4 is generally removed from patient 8. As can be seen in FIG. 6, which depicts patient interface 4 in its free state, at least a portion of one of first and second engagement elements 24 and 28 overlies (from the perspective of FIG. 6) at least a portion of the other of first and second engagement elements 24 and 28. FIG. 5 is intended to depict an intermediate position between the donned state and the free state and demonstrates that frame 20 is of a generally spiral shape.

That is, frame 20 is configured to have a spiral, i.e., helical, free state shape and to have pivotable (or otherwise movable) connections with first and second engagement elements 24 and 28. When placed into its compact free state depicted generally in FIG. 6, patient interface 4 takes up a relatively small amount of space, which is desirable for packing, such as for traveling. When patient interface 4 is returned from its free state (as in FIG. 6) to its expanded state (as in FIG. 4 but immediately prior to installation on patient 8), the spiral shape of frame 20 provides to it the residual stresses that exist in frame 20 when first and second engagement elements 24 and 28 are separated from one another in preparation of installation of patient interface 4 on patient 8.

An improved patient interface 104 in accordance with a second embodiment of the present invention is depicted generally in FIGS. 7 and 8. Patient interface 104 is similar to patient interface 4 in that it includes a cushion 112 connected with a headgear 116 and can generally be said to possess the other features possessed by patient interface 4. However, patient interface 104 employs a first engagement element 124 and a second engagement element 128 that are different than their counterparts in patient interface 4. In particular, first engagement element 124 employs a circumferential brace 144 having an opening 148 formed therein, although circumferential brace 144 is depicted as being of a taller thin-walled structure compared with first engagement element 24. Second engagement element 128 likewise includes a circumferential brace 152 having an opening 156 formed therein that is of a similar configuration to circumferential brace 144 and opening 148. First and second engagement elements 124 and 128 additionally each have a cover 182 and 184, respectively, disposed on circumferential brace 144 and 152 and that extends across opening 148 and 156, respectively. First and second engagement elements 124 and 128 are thus each configured as an enclosure that extends about and covers an ear of patient 8 and which still engages the head of patient 8 with circumferential braces 144 and 152.

Patient interface 104 may be preferred by patient 8 depending upon the feel of the engagement of first and second engagement elements 124 and 128 with the head of patient 8, and/or such preference may be based upon the different acoustic properties that result from the addition of covers 182 and 184 on first and second engagement elements 124 and 128, respectively, by way of example.

An improved patient interface 204 in accordance with a third embodiment of the present invention is indicated generally in FIG. 9. Patient interface 204 is similar to patient interfaces 4 and 104 in that it includes a cushion 212, a headgear 216, and other features thereof, and it performs many of the same functions as patient interfaces 4 and 104. However, patient interface 204 employs first and second engagement elements 224 and 228 that engage the ears of patient 8 rather than engaging the head of patient 8. Frictional forces between patient 8 and first and second engagement elements 224 and 228 thus result from friction between the skin of the ears of patient 8 and first and second engagement elements 224 and 228.

Patient interface 204 may be preferred by patient 8 depending upon the feel of the engagement of first and second engagement elements 224 and 228 with the ears of patient 8, and/or such preference may be based upon the different acoustic properties that result from the engagement of first and second engagement elements 224 and 228 with ears, by way of example.

An improved patient interface 304 in accordance with a fourth embodiment of the present embodiment is indicated generally in FIG. 10. Patient interface 304 is similar to patient interface 204 in that it includes a cushion 312 mounted on a headgear 316 and other features thereof. However, first and second engagement elements 324 and 328 of patient interface 304 each have a receptacle 386 formed therein. Moreover, a pad 388 is received in each such receptacle 386, and pads 388 engage the ears of patient 8. Pads 388 can be formed of any flexible and resilient member that is comfortable, such as a foam filled structure and the like. Pads 388 can potentially provide greater comfort to patient 8 than patient interface 204. It is also understood, however, that patient interface 204 may nevertheless be preferred by patient 8 if the relatively more fixed connection between patient interface 204 and the ears of patient 8 is more desired by patient 8.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface structured to deliver a flow of breathing gas to the airways of a patient, the patient interface comprising:
a cushion structured to be in fluid communication with the airways of the patient and to supply the flow of breathing gas to the airways of the patient; and
a headgear comprising a frame and a pair of engagement elements, the cushion being disposed on the frame, the pair of engagement elements being disposed on the frame and being structured to be engaged with the patient, the engagement elements engaged with the patient each being structured to apply an engagement force in a direction generally toward the patient, the engagement forces being directed generally toward one another;
the frame comprising a base and a pair of legs, the legs extending from the base, the cushion being situated on the base, the pair of engagement elements being disposed on the pair of legs; and
the pair of engagement elements each comprising a circumferential brace having an opening formed therein, the circumferential braces each being structured to engage the head of the patient and to extend circumferentially about at least a portion of one of the ears of the patient, the openings each being structured to receive at least a portion of one of the ear.

2. (canceled)

3. The patient interface of claim 1, wherein the cushion is engaged with the nose of the patient, and wherein the engagement forces are structured to result in frictional forces between the engagement elements and the skin of the patient that are of sufficient magnitude to resist the cushion from becoming disengaged with the nose of the patient.

4. (canceled)

5. The patient interface of claim 1, wherein the pair of engagement elements each further comprise a cover situated on the circumferential brace and extending across at least a portion of the opening, the covers being structured to overlie the ears of the patient.

6. The patient interface of claim 1, wherein the pair of engagement elements each further comprise an audio transducer structured to generate an audible output responsive to a signal.

7. The patient interface of claim 1, wherein the pair of engagement elements each further comprise another audio transducer structured to generate another signal responsive to an audible input, the headgear further comprising a processor apparatus that is structured to receive the another signal and to generate as the signal a noise cancellation signal responsive at least in part to the another signal.

8. The patient interface of claim 1, wherein the frame comprises a wireless transceiver structured to connect wirelessly with a remote wireless device.

9. The patient interface of claim 1, wherein the pair of engagement elements are each structured to engage at least a portion of one of the ears of the patient.

10. The patient interface of claim 9, wherein the pair of engagement elements each comprise a pad element structured to engage at least a portion of one of the ears of the patient.

11. The patient interface of claim 10, wherein the pair of engagement elements each have a receptacle formed therein, the pad elements being situated in the receptacles.

12. The patient interface of claim 1, wherein the legs are each structured to extend generally between a nasal region of the patient and an otological region of the patient.

13. The patient interface of claim 1, wherein the frame is movable between a free state in a condition removed from the patient and a donned state in a condition with the engagement elements engaged with the patient, at least a portion of the frame in the free state being of a generally spiral configuration.

14. The patient interface of claim 13, wherein at least a portion of an engagement element of the pair of engagement elements overlies at least a portion of another engagement element of the pair of engagement elements.

15. The patient interface of claim 13, wherein the pair of engagement elements are movably disposed on the frame.
